SEQUENCE LISTING (2 sequences)

SEQ ID NO: 1 - protein, 378 aa, Drosophila melanogaster
MTMSTNNCESMTSYFTNSYMGADMHHGHYPGNGVTDLDAQQMHHYSQNANHQGNMPYPRFPPYDRMPYYNGQGMDQQQQHQVYSRPDSPSSQVGGVMPQAQTNGQLGVPQQQQQQQQQPSQNQQQQQAQQAPQQLQQQLPQVTQQVTHPQQQQQQPVVYASCKLQAAVGGLGMVPEGGSPPLVDQMSGHHMNAQMTLPHHMGHPQAQLGYTDVGVPDVTEVHQNHHNMGMYQQQSGVPPVGAPPQGMMHQGQGPPQMHQGHPGQHTPPSQNPNSQSSGMPSPLYPWMRSQFGKCQERKRGRQTYTRYQTLELEKEFHFNRYLTRRRRIEIAHALCLTERQIKIWFQNRRMKWKKENKTKGEPGSGGEGDEITPPNSPQ

SEQ ID NO: 2 - protein, 16 aa, Artificial Sequence
  Synthetic construct
RQLKIWFQNRRMKWKK

(19) United States
(12) Patent Application Publication
(10) Pub. No.: US 2010/0190691 A1
(43) Pub. Date: Jul. 29, 2010

(54) DELIVERY OF NUCLEIC ACIDS USING CELL-PENETRATING PEPTIDES

(75) Inventors: Agamemnon A. Epenetos, London (GB); Christina Kousparou, Nicosia (CY)

Correspondence Address: DLA PIPER LLP (US), 4365 EXECUTIVE DRIVE, SUITE 1100, SAN DIEGO, CA, 92121-2133, US

(73) Assignee: Trojan Technologies, Ltd, London (GB)

(21) Appl. No.: 12/694,122

(22) Filed: Jan. 26, 2010

Related U.S. Application Data
(60) Provisional application No. 61/147,724, filed on Jan. 27, 2009.

(51) Int. Cl. A61K 38/16; C07K 9/00; C12N 5/02

(52) U.S. Cl. 514/8; 530/322; 435/325

(57) ABSTRACT

The present invention is based on the innovative concept of conjugating a cell-penetrating peptide (CPP), including a protein transduction domain, to a nucleic acid molecule to provide a nucleic acid-protein conjugate exhibiting enhanced cellular uptake. Accordingly, the invention provides a method of producing a cell permeable nucleic acid molecule conjugate nucleic acid including a nucleic acid conjugated with a homeodomain of an antennapedia homeotic transcription factor protein (Antp), or functional fragment thereof. The invention further provides compositions and methods treating a subject using the conjugates produced by the method described herein.

### RELATED APPLICATION DATA

This application claims the benefit of priority under 35 U.S.C. §119(e) of U.S. Ser. No. 61/147,724, filed Jan. 27, 2009, the entire content of which is incorporated herein by reference.

### BACKGROUND OF THE INVENTION

1. Field of the Invention

The invention relates generally to delivery of molecules to cells and more specifically to delivery of siRNA molecules to cells.

2. Background Information

siRNAs are small, double stranded RNAs, typically 21-23 base pairs in length, that are involved in gene silencing through degradation of mRNA and compacting DNA thereby blocking transcription.

Recently, an increasing number of studies have suggested the potential use of siRNAs as therapeutic tools to knock down protein expression. Due to their hydrophilic nature, they will not be readily internalised by cells. For the majority of eukaryotic cells, the siRNA has to be actively delivered over the plasma membrane by a carrier.

In dividing cells, transfection of siRNA has a maximum effect 2-3 days after transfection, with a knock down lasting for approximately 1 week. In non-dividing cells, the effect can last for several weeks. Vector systems used include adenoviruses, adeno-associated viruses, oncoretroviruses or lentiviruses. Vectors based on cationic lipids include the commercially available lipofectamine. Other delivery strategies include electroporation, calcium phosphate coprecipitation and microinjection.

siRNA can be alternatively delivered using cell-penetrating peptides (CPPs). There are two methods whereby this can be achieved: coincubation/non-covalent siRNA-CPP complex formation or covalent coupling via disulphide bridging. Non-covalent complex formation is simple and involves mixing the CPP with the siRNA prior to addition to the cells. This method relies on electrostatic interactions in which the positively charged CPPs surround the siRNA, masking its negative charges. The fact that these complexes vary in size depending on the molar ratios of the components and the equilibrium between non-bound and on-bound CPPs makes them inappropriate for therapeutic applications, where a defined complex size is required. Thus, covalent coupling possibly offers better solutions. In such a case, there is a defined molecule in which one CPP is bound to one siRNA molecule. It is also more cost-effective since a lot less peptide is used, and less toxic as some CPPs show toxicity in high concentrations.

The formation of a disulphide bridge can be achieved between the CPP and the 5′ end of one strand of the siRNA by using the thiol oxidising agent diamide. The siRNA with a free thiol and the CPP with an N-terminal cysteine is mixed with diamide and incubated for 1 hr prior to being applied to cells. The yield of this coupling technique is approximately 80%. Alternatively, modification of the peptide can be achieved using pyridylhiol which offers higher reactivity with the 5′ thiol on the siRNA. Before performing this disulphide reaction, the siRNA solution must be pretreated with equimolar amounts of tris(2-carboxyethyl)phosphine to reduce the 5′ thiol on the siRNA, after which the CPP is added with pyridylhiol to form a disulphide bridge. The estimated yield of this protocol is 90%.

The antennapedia homeodomain is a sequence-specific transcription factor from the organism Drosophila melanogaster. This protein is encoded by the antennapedia (antp) gene. Antp is a member of a regulatory system that gives cells specific positions on the anterior-posterior axis of the organism. Thus, Antp aids in the control of cell development in the mesothorax segment in Drosophila. The homeobox domain, or homeodomain, is one that binds DNA through a helix-turn-helix structural motif. Proteins that contain a homeobox domain usually play a role in development, and many of these are sequence-specific transcription factors such as Antp. The relationship between the functional complexity and the molecular organization of the antennapedia locus of Drosophila melanogaster are becoming better known. For example, expression and function of the homoeotic genes antennapedia and sex combs is reduced in the embryonic midgut of Drosophila.

The antennapedia homeodomain is approximately 68 amino acid residues long as shown underlined in FIG. 7. Antennapedia is a universal delivery CPP that has been shown to target 100% of cells in a non-toxic, temperature-, energy- and receptor-independent manner. It therefore represents the ideal tool for the delivery of exogenous siRNA in RNA-interference therapeutic development. Some of the advantages include:
1) simple production, upscalable;
2) cost effective;
3) efficient, non-viral delivery;
4) non-traumatic membrane trafficking;
5) high transduction efficiencies in difficult-to-transfect cell types;
6) proven delivery to all organs, including the brain from blood following intravenous administration with an intact blood-brain barrier;
7) established preclinical efficacy of Antp-mediated products;
8) established pharmacokinetics with clearance characteristics resembling those of small molecules;
9) optimized protocols for in vitro and in vivo administrations available;
10) same protocol for different cell lines and animals allows easy switch between models;
11) non-reagent-based transfection;
12) high reproducibility; and
13) established no reagent-induced immunogenicity.

Researchers observed that double stranded RNA (“dsRNA”) could be used to inhibit protein expression. This ability to silence a gene has broad potential for treating human diseases, and many researchers and commercial entities are currently investing considerable resources in developing therapies based on this technology.

Double stranded RNA induced gene silencing can occur on at least three different levels: (i) transcription inactivation, which refers to RNA guided DNA or histone methylation; (ii) siRNA induced mRNA degradation; and (iii) mRNA induced transcriptional attenuation.

It is generally considered that the major mechanism of RNA induced silencing (RNA interference, or RNAi) in mammalian cells is mRNA degradation. Initial attempts to use RNAi in mammalian cells focused on the use of long strands of dsRNA. However, these attempts to induce RNAi met with limited success, due in part to the induction of the interferon response, which results in a general, as opposed to a target-specific, inhibition of protein synthesis. Thus, long dsRNA is not a viable option for RNAi in mammalian systems.

More recently it has been shown that when short (18-30 bp) RNA duplexes are introduced into mammalian cells in culture, sequence-specific inhibition of target mRNA can be realized without inducing an interferon response. Certain of these short dsRNAs, referred to as small inhibitory RNAs (“siRNAs”), can act catalytically at sub-molar concentrations to cleave greater than 95% of the target mRNA in the cell. A description of the mechanisms for siRNA activity, as well as some of its applications have been described in the literature.

From a mechanistic perspective, introduction of long double stranded RNA into plants and invertebrate cells is broken down into siRNA by a Type II endonuclease known as Dicer. Dicer, a ribonuclease-III-like enzyme, processes the dsRNA into 19-23 base pair short interfering RNAs with characteristic two base 3′ overhangs. The siRNAs are then incorporated into an RNA-induced silencing complex (RISC) where one or more helicases unwind the siRNA duplex, enabling the complementary antisense strand to guide target recognition. Upon binding to the appropriate target mRNA, one or more endonucleases within the RISC cleaves the target to induce silencing.

The interference effect can be long lasting and may be detectable after many cell divisions. Moreover, RNAi exhibits sequence specificity. Thus, the RNAi machinery can specifically knock down one type of transcript, while not affecting closely related mRNA. These properties make siRNA a potentially valuable tool for inhibiting gene expression and studying gene function and drug target validation. Moreover, siRNAs are potentially useful as therapeutic agents against: (1) diseases that are caused by over-expression or misexpression of genes; and (2) diseases brought about by expression of genes that contain mutations.

Successful siRNA-dependent gene silencing depends on a number of factors. One of the most contentious issues in RNAi is the question of the necessity of siRNA design, i.e., considering the sequence of the siRNA used. Early work in C. elegans and plants circumvented the issue of design by introducing long dsRNA. In this primitive organism, long dsRNA molecules are cleaved into siRNA by Dicer, thus generating a diverse population of duplexes that can potentially cover the entire transcript. While some fraction of these molecules are non-functional (i.e., induce little or no silencing) one or more have the potential to be highly functional, thereby silencing the gene of interest and alleviating the need for siRNA design. Unfortunately, due to the interferon response, this same approach is unavailable for mammalian systems. While this effect can be circumvented by bypassing the Dicer cleavage step and directly introducing siRNA, this tactic carries with it the risk that the chosen siRNA sequence may be non-functional or semi-functional.

RNA interference (RNAi) is a process by which double-stranded RNA (dsRNA) is used to silence gene expression. While not wanting to be bound by theory, RNAi begins with the cleavage of longer dsRNAs into small interfering RNAs (siRNAs) by an RNaseIIl-like enzyme, dicer. SiRNAs are dsRNAs that are usually about 19 to 28 nucleotides, or 20 to 25 nucleotides, or 21 to 22 nucleotides in length and often contain 2-nucleotide 3′ overhangs, and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. Therefore, the siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA design (e.g., decreased siRNA duplex stability at the 5′ end of the desired guide strand) can favor incorporation of the desired guide strand into RISC.

The antisense strand of an siRNA is the active guiding agent of the siRNA in that the antisense strand is incorporated into RISC, thus allowing RISC to identify target mRNAs with at least partial complementarity to the antisense siRNA strand for cleavage or translational repression. RISC-mediated cleavage of mRNAs having a sequence at least partially complementary to the guide strand leads to a decrease in the steady state level of that mRNA and of the corresponding protein encoded by this mRNA. Alternatively, RISC can also decrease expression of the corresponding protein via translational repression without cleavage of the target mRNA.

### SUMMARY OF THE INVENTION

The present invention is based on the innovative concept of conjugating a cell-penetrating peptide (CPP), including a protein transduction domain to a nucleic acid molecule to provide for efficient delivery of the conjugate into a cell.

Accordingly, in one aspect, the invention provides a method of producing a cell permeable nucleic acid molecule conjugate. The method includes conjugating a nucleic acid with a cell-penetrating peptide (CPP), the CPP including a homeodomain of an antennapedia homeotic transcription factor protein (Antp), or functional fragment thereof. Conjugation is performed as provided in Schemes 1-6 (FIGS. 1-6), or combinations thereof.

In another aspect, the invention provides a cell permeable nucleic acid molecule conjugate. The cell permeable nucleic acid molecule conjugate includes a nucleic acid molecule; and a cell-penetrating peptide (CPP), the CPP comprising a homeodomain of an antennapedia homeotic transcription factor protein (Antp), or functional fragment thereof.

In another aspect, the invention provides a method of introducing a cell permeable nucleic acid molecule conjugate into a cell. The method includes producing a cell permeable nucleic acid molecule conjugate using the method of the invention; and contacting a cell with the conjugate, thereby introducing the cell permeable nucleic acid molecule conjugate into the cell. In various embodiments, contacting may be performed in vivo or in vitro.

In another aspect, the invention provides a method of treating a subject in need thereof. The method includes administering to the subject a cell permeable nucleic acid molecule conjugate of the present invention.

In another aspect, the invention provides a pharmaceutical composition including the cell permeable nucleic acid molecule conjugate produced by the method described herein.

In various embodiments, the nucleic acid molecule conjugate includes a double stranded RNA, such as siRNA. In a related embodiment, the nucleic acid molecule conjugate includes a CPP derived from SEQ ID NO: 1. For example the CPP includes at least 5 contiguous amino acid residues of SEQ ID NO: 1 from residue 283 to 356, such as SEQ ID NO: 2. In yet another embodiment, the nucleic acid molecule conjugate includes greater than 1 nucleic acid molecule conjugated to a single CPP.

### BRIEF DESCRIPTION OF THE DRAWINGS

FIG. 1 is a diagram showing a conjugation scheme in one embodiment of the invention.

FIG. 2 is a diagram showing a conjugation scheme in one embodiment of the invention.

FIG. 3 is a diagram showing a conjugation scheme in one embodiment of the invention.

FIG. 4 is a diagram showing a conjugation scheme in one embodiment of the invention.

FIG. 5 is a diagram showing a conjugation scheme in one embodiment of the invention.

FIG. 6 is a diagram showing a conjugation scheme in one embodiment of the invention.

FIG. 7 is a graphic representation of the amino acid sequence of antennapedia homeotic transcription factor protein (Antp) (SEQ ID NO: 1).

### DETAILED DESCRIPTION OF THE INVENTION

The present invention is based on the innovative concept of conjugating a cell-penetrating peptide (CPP), including a protein transduction domain, to a nucleic acid molecule to provide a nucleic acid-protein conjugate exhibiting enhanced cellular uptake. The conjugate molecule gains entry into a cell via the protein transduction domain. The protein transduction domain of antennapedia (Antp) homeotic transcription factor has the ability to transduce or travel through biological membranes independent of classical receptor- or endocytosis-mediated pathways. Thus the conjugate molecule exhibits enhanced cellular uptake and serves as an efficient method for targeting and delivering nucleic acids, such as siRNA's to a cell.

Before the present composition, methods, and culturing methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure. All publications mentioned herein are incorporated herein by reference in their entirety.

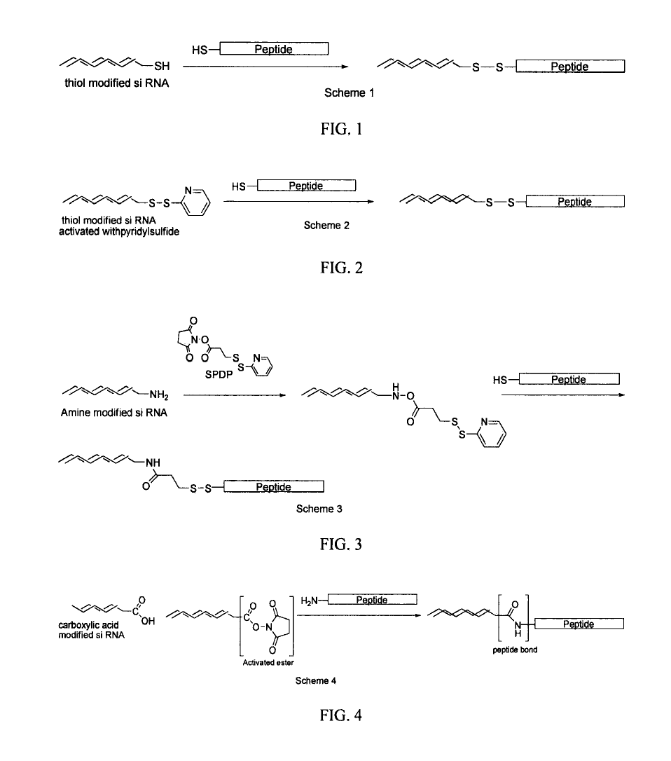

In one aspect, the present invention provides a method of producing a cell permeable nucleic acid molecule conjugate. Essentially, the method includes conjugating one or more nucleic acid molecules with a CPP using reaction Schemes 1-6 (FIGS. 1-6) or combination thereof. As discussed herein, the CPP comprises a homeodomain of an antennapedia homeotic transcription factor protein (Antp), or functional fragment thereof.

In various embodiments, the nucleic acid molecule is conjugated to a CPP using reaction Schemes 1-6 (FIGS. 1-6), depending in part on the chemistry of the 3′ or 5′ end of the nucleic acid molecule. In one embodiment, a nucleic acid molecule chemically modified at the 3′ or 5′ end with a thiol group is conjugated as follows. As shown in Scheme 1 (FIG. 1), conjugating includes contacting the nucleic acid molecule having a thiol group at its 3′ or 5′ end with the CPP which includes an amino acid residue including a thiol group, in the presence of a thiol crosslinking agent. Typically the amino acid residue including a thiol group is cysteine, however, the amino acid may be any residue chemically modified to include a thiol group. Various thiol crosslinking agents are known in the art and suitable for use in the present invention. In an exemplary embodiment, the thiol crosslinking agent is diamine.

In a related embodiment, as shown in Scheme 2, (FIG. 2) the thiol group of the nucleic acid molecule is contacted with a pyridyl sulfide before contacting the nucleic acid molecule with the CPP. This achieves activation of the thiol containing component of the nucleic acid molecule before addition of the peptide, which allows specific heterodisulfide formation without concomitant homodisulfide formation of the peptide or nucleic acid component. Various pyridyl sulfides are known in the art and suitable for use in the present invention.

In a related embodiment, as shown in Scheme 3 (FIG. 3) conjugation of a 3′ or 5′ amino modified nucleic acid with a CPP may be achieved. The method includes contacting the nucleic acid molecule having an amine group at its 3′ or 5′ end, with an amino and thiol reactive heterobifunctional crosslinking agent. The nucleic acid is subsequently contacted with a CPP including an amino acid residue including a thiol group in the presence of a thiol crosslinking agent. As discussed herein, typically the amino acid residue including a thiol group is cysteine, however, the amino acid may be any residue chemically modified to include a thiol group. Various amino and thiol reactive heterobifunctional crosslinking agent are known in the art and suitable for use in the present invention. In an exemplary embodiment, the amino and thiol reactive heterobifunctional crosslinking agent is N-succinimidyl-3-(2-pyridyldithio)propionate (SPDP) and the thiol crosslinking agent is diamide.

In a related embodiment, the multiple nucleic acid molecules may be conjugated to a single CPP. As shown in Scheme 4, (FIG. 4) this is achieved by first contacting a nucleic acid molecule including a 3′ or 5′ terminal carboxylic acid group that has been activated using carbodiimide/N-hydroxy-succinimide chemistry to produce an activated ester. The nucleic acid is subsequently contacted with a CPP including an amino acid residue having an amine group. Typically the amino acid residue including an amine group is lysine, however, the amino acid may be any residue chemically modified to include an amine group.

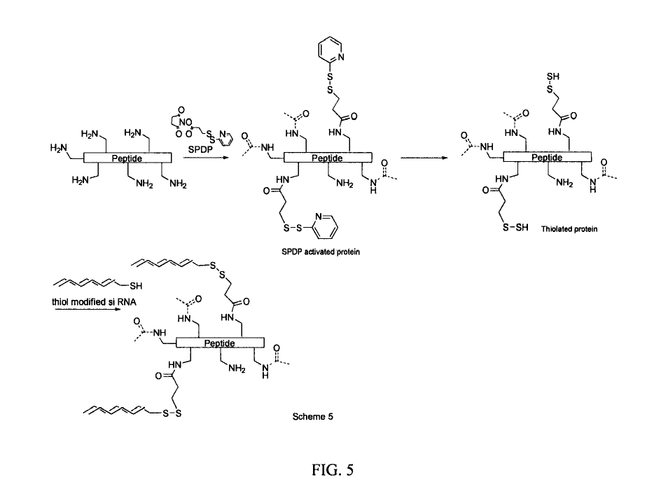

In a related embodiment, the CPP may be thiolated using an amino and thiol reactive heterobifunctional crosslinking agent to convert the amine groups contained on amino acid residues, such as lysine, to thiol groups as shown in Scheme 5 (FIG. 5). This embodiment includes first contacting the CPP including an amino acid residue comprising an amine group, with an amino and thiol reactive heterobifunctional crosslinking agent. The CPP is then contacted with a disulfide reducing agent to release the pyridine-2-thione leaving group to form a free sulfhydryl (thiol) group. Subsequently, the CPP is contacted with a nucleic acid molecule including a thiol group at its 3′ or 5′ end in the presence of a thiol crosslinking agent. While one of skill in the art would understand that various crosslinking agents may be used and are commonly known in the art, in an exemplary embodiment, the amino and thiol reactive heterobifunctional crosslinking agent is N-succinimidyl-3-(2-pyridyldithio)propionate (SPSP), the disulfide reducing agent of is dithiothreitol (DTT), and the thiol crosslinking agent of is diamide.

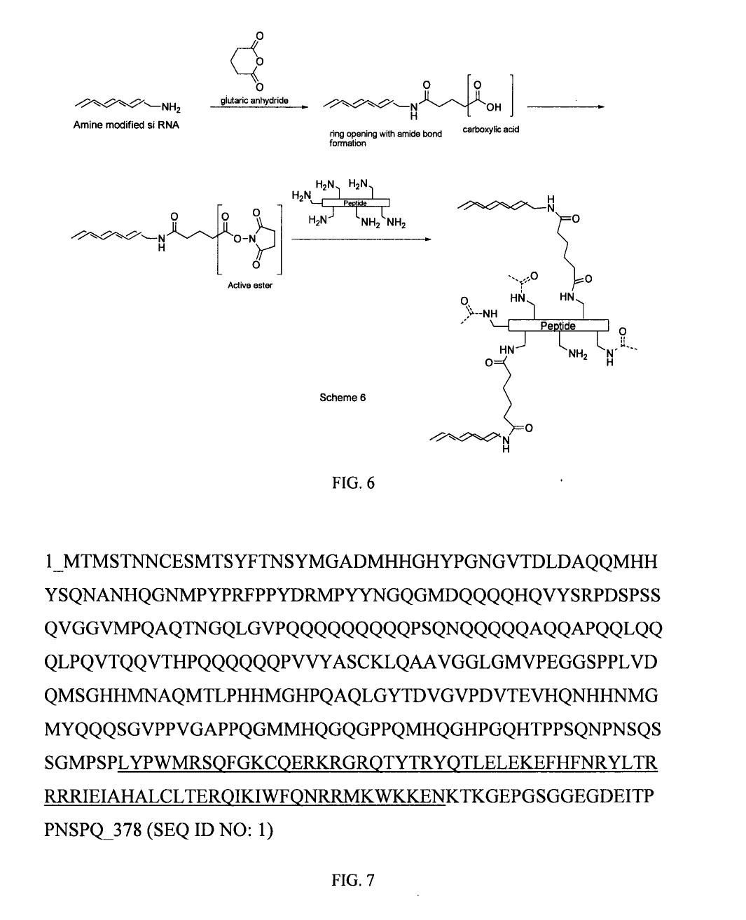

In a related embodiment, a nucleic acid molecule including a 3′ or 5′ terminal amine group may be reacted with an agent to convert the amine group to a carboxylic acid and subsequently converting the acid to an activated ester for conjugation with the CPP. As shown in Scheme 6 (FIG. 6), the method includes contacting a nucleic acid molecule having an amine group at its 3′ or 5′ end with a reagent to convert the amine group to a carboxylic acid, such as glutaric anyhdride. The nucleic acid is then contacted with a second reagent to convert the carboxylic acid to an active ester, for example by treatment with amino and thiol reactive heterobifunctional crosslinking agent, such as N-succinimidyl-3-(2-pyridyldithio)propionate (SPSP). Finally, the nucleic acid molecule is contacted with a CPP including an amino acid residue having an amine group, such as lysine.

In another aspect, the present invention provides a cell permeable nucleic acid molecule conjugate produced using the method described herein. The cell permeable nucleic acid molecule conjugate includes a nucleic acid molecule; and a cell-penetrating peptide (CPP), the CPP comprising a homeodomain of an antennapedia homeotic transcription factor protein (Antp), or functional fragment thereof.

The conjugate molecules of the present invention gain entry into a cell via the CPP, also known as a protein transduction domain. The CPP of antennapedia (Antp) homeotic transcription factor has the ability to transduce or travel through biological membranes independent of classical receptor- or endocytosis-mediated pathways. The CPP of antennapedia (Antp) homeotic transcription factor is incorporated into the conjugate of the present invention to successfully transport the nucleic acid-protein conjugate into a cell. In an exemplary embodiment, the CPP is from an antennapedia (Antp) homeotic transcription factor, or functional fragments thereof. For example, the CPP is derived from SEQ ID NO: 1, more particularly, the homeodomain corresponding to amino acid residues 283-356 of SEQ ID NO: 1 as shown underlined in FIG. 7 (Accession No. P02833). In an exemplary embodiment, the CPP includes a CPP sequence having the following amino acid sequence: RQLKIWFQNRRMKWKK (SEQ ID NO: 2). In various embodiments, the CPP includes at least 5, 6, 7, 8, 9, 10, 11, 12, 13, 14, 15, 16, 17, 18, 19, 20, 25, 30, 35, 40, 45, 50, 55 or 60 contiguous residues of SEQ ID NO: 1 as shown in FIG. 7, so long as the fragment retains transduction activity. One of skill in the art would understand that various isoforms of Antp exist and may be used as CPPs in the present invention. For example, CPPs may be derived from the homeodomains of exemplary sequences, such as those described in NCBI reference sequences: NP_{—}996175.1, NP_{—}996173.1, NP_{—}996172.1, NP_{—}996171.1, NP_{—}996174.1, NP_{—}996170.1, NP_{—}996169.1, NP_{—}996166.1, NP_{—}996168.1, NP_{—}996167.1, and NP_{—}996176.1.

The type and size of the CPP will be guided by several parameters including the extent of transduction desired. Typically the CPP will be capable of transducing at least about 20%, 25%, 50%, 75%, 80% or 90%, 95%, 98% and up to, and including, about 100% of the cells. Transduction efficiency, typically expressed as the percentage of transduced cells, can be determined by several conventional methods known in the art. One of skill in the art would understand that any function fragment of the CPP domain of antennapedia (Antp) homeotic transcription factor may be used in the present invention so long as the functional fragment retains protein transduction activity.

A polypeptide (including a CPP polypeptide) refers to a polymer in which the monomers are amino acid residues which are joined together through amide bonds. When the amino acids are alpha-amino acids, either the L-optical isomer or the D-optical isomer can be used. A polypeptide encompasses an amino acid sequence and includes modified sequences such as glycoproteins, retro-inverso polypeptides, D-amino acid modified polypeptides, and the like. A polypeptide includes naturally occurring proteins, as well as those which are recombinantly or synthetically synthesized. A polypeptide may comprise more than one domain having a function that can be attributed to the particular fragment or portion of a polypeptide. A domain, for example, includes a portion of a polypeptide which exhibits at least one useful epitope or functional domain. Two or more domains may be functionally linked such that each domain retains its function yet comprises a single polypeptide (e.g., a fusion polypeptide). For example, a functional fragment of a CPP includes a fragment which retains transduction activity. Biologically functional fragments, for example, can vary in size from a polypeptide fragment as small as an epitope capable of binding an antibody molecule, to a large polypeptide capable of participating in the characteristic induction or programming of phenotypic changes within a cell.

Polypeptides and fragments can have the same or substantially the same amino acid sequence as the naturally derived polypeptide or domain. “Substantially identical” means that an amino acid sequence is largely, but not entirely, the same, but retains a functional activity of the sequence to which it is related. An example of a functional activity is that the fragment is capable of transduction. In general two polypeptides or domains are “substantially identical” if their sequences are at least 85%, 90%, 95%, 98% or 99% identical, or if there are conservative variations in the sequence.

A polypeptide of the disclosure can be composed of amino acids joined to each other by peptide bonds or modified peptide bonds, e.g., peptide isosteres, and may contain amino acids other than the 20 gene-encoded amino acids. The polypeptides may be modified by either natural processes, such as posttranslational processing, or by chemical modification techniques which are well known in the art. Such modifications are well described in the art. Modifications can occur anywhere in a peptide or polypeptide, including the peptide backbone, the amino acid side-chains and the amino or carboxyl termini. It will be appreciated that the same type of modification may be present in the same or varying degrees at several sites in a given peptide or polypeptide. Also, a given peptide or polypeptide may contain many types of modifications. A peptide or polypeptide may be branched, for example, as a result of ubiquitination, and they may be cyclic, with or without branching. Cyclic, branched, and branched cyclic peptides and polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

As discussed herein, the conjugates of the present invention include a nucleic acid molecule conjugated to an antennapedia cell-penetrating peptide (CPP). The invention further provides a method for introducing a cell permeable nucleic acid molecule conjugate into a cell. The method includes producing a cell permeable nucleic acid molecule conjugate using the method of the invention; and contacting a cell with the conjugate, thereby introducing the cell permeable nucleic acid molecule conjugate into the cell. In various embodiments, contacting may be performed in vivo or in vitro.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

In exemplary aspects, the conjugates of the present invention include a nucleic acid molecule that is RNA, such as a double stranded RNA, for example siRNA. The terms “small interfering RNA” and “siRNA” are used herein to refer to short interfering RNA or silencing RNA, which are a class of short double-stranded RNA molecules that play a variety of biological roles. Most notably, siRNA is involved in the RNA interference (RNAi) pathway where the siRNA interferes with the expression of a specific gene. In addition to their role in the RNAi pathway, siRNAs also act in RNAi-related pathways (e.g., as an antiviral mechanism or in shaping the chromatin structure of a genome). Interfering RNAs of the invention appear to act in a catalytic manner for cleavage of target mRNA, e.g., interfering RNA is able to effect inhibition of target mRNA in substoichiometric amounts. As compared to antisense therapies, significantly less interfering RNA is required to provide a therapeutic effect under such cleavage conditions.

Single-stranded interfering RNAs can be synthesized chemically or by in vitro transcription or expressed endogenously from vectors or expression cassettes as described herein in reference to double-stranded interfering RNAs. 5′ Phosphate groups may be added via a kinase, or a 5′ phosphate may be the result of nuclease cleavage of an RNA. A hairpin interfering RNA is a single molecule (e.g., a single oligonucleotide chain) that comprises both the sense and antisense strands of an interfering RNA in a stem-loop or hairpin structure (e.g., a shRNA). For example, shRNAs can be expressed from DNA vectors in which the DNA oligonucleotides encoding a sense interfering RNA strand are linked to the DNA oligonucleotides encoding the reverse complementary antisense interfering RNA strand by a short spacer. If needed for the chosen expression vector, 3′ terminal T's and nucleotides forming restriction sites may be added. The resulting RNA transcript folds back onto itself to form a stem-loop structure.

Techniques for selecting target sequences for siRNAs are provided, for example, by Tuschl, T. et al., “The siRNA User Guide,” revised May 6, 2004, available on the Rockefeller University web site; by Technical Bulletin #506, “siRNA Design Guidelines,” Ambion Inc. at Ambion's web site; and by other web-based design tools at, for example, Life Technologies, Dharmacon, Integrated DNA Technologies, Genscript, or Proligo web sites. Initial search parameters can include G/C contents between 35% and 55% and siRNA lengths between 19 and 27 nucleotides. The target sequence may be located in the coding region or in the 5′ or 3′ untranslated regions of the mRNA. The target sequences can be used to derive interfering RNA molecules, such as those described herein.

The target RNA cleavage reaction guided by siRNAs and other forms of interfering RNA is highly sequence specific. For example, in general, an siRNA molecule contains a sense nucleotide strand identical in sequence to a portion of the target mRNA and an antisense nucleotide strand exactly complementary to a portion of the target for inhibition of mRNA expression. However, 100% sequence complementarity between the antisense siRNA strand and the target mRNA, or between the antisense siRNA strand and the sense siRNA strand, is not required to practice the present invention, so long as the interfering RNA can recognize the target mRNA and silence expression. Thus, for example, the invention allows for sequence variations between the antisense strand and the target mRNA and between the antisense strand and the sense strand, including nucleotide substitutions that do not affect activity of the interfering RNA molecule, as well as variations that might be expected due to genetic mutation, strain polymorphism, or evolutionary divergence, wherein the variations do not preclude recognition of the antisense strand to the target mRNA.

Polynucleotides of the present invention, such as RNA molecules may be of any suitable length. For example, one of skill in the art would understand what length are suitable for antisense oligonucleotides or RNA molecule to be used to regulate gene expression. Such molecules are typically from about 5 to 100, 5 to 50, 5 to 45, 5 to 40, 5 to 35, 5 to 30, 5 to 25, 5 to 20, or 10 to 20 nucleotides in length. For example the molecule may be about 5, 10, 15, 16, 17, 18, 19, 20, 21, 22, 23, 24, 25, 26, 27, 28, 29, 30, 31, 32, 33, 34, 35, 40, 45 or 50 nucleotides in length. Such polynucleotides may include from at least about 15 to more than about 120 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides.

In one embodiment of the invention, interfering RNA of the invention has a sense strand and an antisense strand, and the sense and antisense strands comprise a region of at least near-perfect contiguous complementarity of at least 19 nucleotides. In another embodiment of the invention, an interfering RNA of the invention has a sense strand and an antisense strand, and the antisense strand comprises a region of at least near-perfect contiguous complementarity of at least 19 nucleotides to a target sequence, and the sense strand comprises a region of at least near-perfect contiguous identity of at least 19 nucleotides with a target sequence of mRNA. In a further embodiment of the invention, the interfering RNA comprises a region of at least 13, 14, 15, 16, 17, or 18 contiguous nucleotides having percentages of sequence complementarity to or, having percentages of sequence identity with, the penultimate 13, 14, 15, 16, 17, or 18 nucleotides, respectively, of the 3′ end of the corresponding target sequence within an mRNA. The length of each strand of the interfering RNA comprises about 19 to about 49 nucleotides, and may comprise a length of about 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides.

In certain embodiments, the antisense strand of an interfering RNA of the invention has at least near-perfect contiguous complementarity of at least 19 nucleotides with the target mRNA. “Near-perfect,” as used herein, means the antisense strand of the siRNA is “substantially complementary to,” and the sense strand of the siRNA is “substantially identical to” at least a portion of the target mRNA. “Identity,” as known by one of ordinary skill in the art, is the degree of sequence relatedness between nucleotide sequences as determined by matching the order and identity of nucleotides between the sequences. In one embodiment, the antisense strand of an siRNA having 80% and between 80% up to 100% complementarity, for example, 85%, 90% or 95% complementarity, to the target mRNA sequence are considered near-perfect complementarity and may be used in the present invention. “Perfect” contiguous complementarity is standard Watson-Crick base pairing of adjacent base pairs. “At least near-perfect” contiguous complementarity includes “perfect” complementarity as used herein. Computer methods for determining identity or complementarity are designed to identify the greatest degree of matching of nucleotide sequences, for example, BLASTN (Altschul, S. F., et al. (1990) J. Mol. Biol. 215:403-410).

The term “percent identity” describes the percentage of contiguous nucleotides in a first nucleic acid molecule that is the same as in a set of contiguous nucleotides of the same length in a second nucleic acid molecule. The term “percent complementarity” describes the percentage of contiguous nucleotides in a first nucleic acid molecule that can base pair in the Watson-Crick sense with a set of contiguous nucleotides in a second nucleic acid molecule.

The relationship between a target mRNA and one strand of an siRNA (the sense strand) is that of identity. The sense strand of an siRNA is also called a passenger strand, if present. The relationship between a target mRNA and the other strand of an siRNA (the antisense strand) is that of complementarity. The antisense strand of an siRNA is also called a guide strand.

The sense and antisense strands in an interfering RNA molecule can also comprise nucleotides that do not form base pairs with the other strand. For example, one or both strands can comprise additional nucleotides or nucleotides that do not pair with a nucleotide in that position on the other strand, such that a bulge or a mismatch is formed when the strands are hybridized. Thus, an interfering RNA molecule of the invention can comprise sense and antisense strands having mismatches, G-U wobbles, or bulges. Mismatches, G-U wobbles, and bulges can also occur between the antisense strand and its target (see, for example, Saxena et al., 2003, J. Biol. Chem. 278:44312-9).

One or both of the strands of double-stranded interfering RNA may have a 3′ overhang of from 1 to 6 nucleotides, which may be ribonucleotides or deoxyribonucleotides or a mixture thereof. The nucleotides of the overhang are not base-paired. In one embodiment of the invention, the interfering RNA comprises a 3′ overhang of TT or UU. In another embodiment of the invention, the interfering RNA comprises at least one blunt end. The termini usually have a 5′ phosphate group or a 3′ hydroxyl group. In other embodiments, the antisense strand has a 5′ phosphate group, and the sense strand has a 5′ hydroxyl group. In still other embodiments, the termini are further modified by covalent addition of other molecules or functional groups.

The sense and antisense strands of the double-stranded siRNA may be in a duplex formation of two single strands as described above or may be a single-stranded molecule where the regions of complementarity are base-paired and are covalently linked by a linker molecule to form a hairpin loop when the regions are hybridized to each other. It is believed that the hairpin is cleaved intracellularly by a protein termed dicer to form an interfering RNA of two individual base-paired RNA molecules. A linker molecule can also be designed to comprise a restriction site that can be cleaved in vivo or in vitro by a particular nuclease.

In one embodiment, the invention provides an interfering RNA molecule that comprises a region of at least 13 contiguous nucleotides having at least 90% sequence complementarity to, or at least 90% sequence identity with, the penultimate 13 nucleotides of the 3′ end of an mRNA corresponding to a DNA target, which allows a one nucleotide substitution within the region. Two nucleotide substitutions (i.e., 11/13=85% identity/complementarity) are not included in such a phrase. In another embodiment, the invention provides an interfering RNA molecule that comprises a region of at least 14 contiguous nucleotides having at least 85% sequence complementarity to, or at least 85% sequence identity with, the penultimate 14 nucleotides of the 3′ end of an mRNA corresponding to a DNA target. Two nucleotide substitutions (e.g., 12/14=86% identity/complementarity) are included in such a phrase. In a further embodiment, the invention provides an interfering RNA molecule that comprises a region of at least 15, 16, 17, or 18 contiguous nucleotides having at least 80% sequence complementarity to, or at least 80% sequence identity with, the penultimate 14 nucleotides of the 3′ end of an mRNA corresponding to a DNA target. Three nucleotide substitutions are included in such a phrase.

The penultimate base in a nucleic acid sequence that is written in a 5′ to 3′ direction is the next to the last base, e.g., the base next to the 3′ base. The penultimate 13 bases of a nucleic acid sequence written in a 5′ to 3′ direction are the last 13 bases of a sequence next to the 3′ base and not including the 3′ base. Similarly, the penultimate 14, 15, 16, 17, or 18 bases of a nucleic acid sequence written in a 5′ to 3′ direction are the last 14, 15, 16, 17, or 18 bases of a sequence, respectively, next to the 3′ base and not including the 3′ base.

Interfering RNAs may be generated exogenously by chemical synthesis, by in vitro transcription, or by cleavage of longer double-stranded RNA with dicer or another appropriate nuclease with similar activity. Chemically synthesized interfering RNAs, produced from protected ribonucleoside phosphoramidites using a conventional DNA/RNA synthesizer, may be obtained from commercial suppliers. Interfering RNAs can be purified by extraction with a solvent or resin, precipitation, electrophoresis, chromatography, or a combination thereof, for example. Alternatively, interfering RNA may be used with little if any purification to avoid losses due to sample processing.

When interfering RNAs are produced by chemical synthesis, phosphorylation at the 5′ position of the nucleotide at the 5′ end of one or both strands (when present) can enhance siRNA efficacy and specificity of the bound RISC complex, but is not required since phosphorylation can occur intracellularly.

Interfering RNAs can also be expressed endogenously from plasmid or viral expression vectors or from minimal expression cassettes, for example, PCR generated fragments comprising one or more promoters and an appropriate template or templates for the interfering RNA. Examples of commercially available plasmid-based expression vectors for shRNA include members of the pSilencer series (Ambion, Austin, Tex.) and pCpG-siRNA (InvivoGen, San Diego, Calif.). Viral vectors for expression of interfering RNA may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCK-iT.TM.-DEST (Invitrogen, Carlsbad, Calif.). Selection of viral vectors, methods for expressing the interfering RNA from the vector and methods of delivering the viral vector are within the ordinary skill of one in the art. Examples of kits for production of PCR-generated shRNA expression cassettes include Silencer Express (Ambion, Austin, Tex.) and siXpress (Minis, Madison, Wis.).

In certain embodiments of the present invention, an antisense strand of an interfering RNA hybridizes with an mRNA in vivo as part of the RISC complex.

“Hybridization” refers to a process in which single-stranded nucleic acids with complementary or near-complementary base sequences interact to form hydrogen-bonded) complexes called hybrids. Hybridization reactions are sensitive and selective. In vitro, the specificity of hybridization (i.e., stringency) is controlled by the concentrations of salt or formamide in prehybridization and hybridization solutions, for example, and by the hybridization temperature; such procedures are well known in the art. In particular, stringency is increased by reducing the concentration of salt, increasing the concentration of formamide, or raising the hybridization temperature.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In various embodiments antisense oligonucleotides or RNA molecules include oligonucleotides containing modifications. A variety of modification are known in the art and contemplated for use in the present invention. For example oligonucleotides containing modified backbones or non-natural internucleoside linkages are contemplated. As used herein, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In various aspects modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Certain oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

In various aspects modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In various aspects, oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. In various aspects, oligonucleotides may include phosphorothioate backbones and oligonucleosides with heteroatom backbones. Modified oligonucleotides may also contain one or more substituted sugar moieties. In some embodiments oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH.sub.₂)ₙOCH₃, O(CH₂)ₙNH₂, O(CH₂)ₙCH₃, O(CH₂)ₙONH₂ and O(CH₂)ₙON[(CH₂)CH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N3, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Another modification includes 2′-methoxyethoxy(2′OCH₂CH₂OCH₃, also known as 2′-O-(2-methoxyethyl) or 2′-MOE).

In related aspects, the present invention includes use of Locked Nucleic Acids (LNAs) to generate antisense nucleic acids having enhanced affinity and specificity for the target polynucleotide. LNAs are nucleic acid in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2.

Other modifications include 2′-methoxy(2′-O—CH₃), 2′-aminopropoxy(2′-OCH₂CH₂CH₂NH₂), 2′-allyl (2′-CH—CH—CH₂), 2′-O-allyl (2′-O—CH₂—CHCH₂), 2′-fluoro (2′-F), 2′-amino, 2′-thio, 2′-Omethyl, 2′-methoxymethyl, 2′-propyl, and the like. The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazi-n-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrimido[3′,′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases are known in the art. Certain of these nucleobases are particularly useful for increasing the binding affinity of the oligomeric compounds described herein. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2 C and are presently preferred base substitutions, even more particularly when combined with 2′-O-methoxyethyl sugar modifications.

Another modification of the antisense oligonucleotides described herein involves chemically linking to the oligonucleotide one or more moieties or conjugates which enhance the activity, cellular distribution or cellular uptake of the oligonucleotide. The antisense oligonucleotides can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., dihexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylaminocarbonyloxycholesterol moiety.

In another aspect, the invention provides a method of treating a subject in need thereof. The method includes administering to the subject a cell permeable nucleic acid molecule conjugate of the present invention. Typically a conjugate molecule described herein will be formulated with a pharmaceutically acceptable carrier, although the conjugate molecule may be administered alone, as a pharmaceutical composition.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, and the like, and primates (including monkeys, chimpanzees, orangutans and gorillas) are included within the definition of subject.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

A pharmaceutical composition described herein can be prepared to include a conjugate molecule of the present invention, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as is well known in the art.

The total amount of a conjugate to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of conjugate to treat a specific disease in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In general, a suitable daily dose of a compound of the invention will be that amount of the compound which is the lowest dose effective to produce a therapeutic effect. Such an effective dose will generally depend upon the factors described above. If desired, the effective daily dose of the active compound may be administered as two, three, four, five, six or more sub-doses administered separately at appropriate intervals throughout the day, optionally, in unit dosage forms. There may be a period of no administration followed by another regimen of administration.

It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The term “effective amount” is defined as the amount of the conjugate or pharmaceutical composition that will elicit the biological or medical response of a tissue, system, animal or human that is being sought by the researcher, veterinarian, medical doctor or other clinician.

The following examples are intended to illustrate but not limit the invention.

### Example 1

### Generation of siRNA Conjugates

By way of example, the 3′- or 5′-terminus of the sense strand is generally used for conjugation. The principal sites of conjugation on antennapedia are the thiol groups of cysteine and the amino group of lysines. In general there are more lysine residues on proteins than cysteine. A brief survey of the literature has shown that the most commonly used and simplest way of chemically conjugating siRNA on to peptides is to link through the thiol group of cysteine. This can be achieved in essentially two ways depending on the modification carried out on the 3′- or 5′-end of the RNA strand. Commercially available siRNA which have been chemically modified at the 3′- or 5′-end with a thiol group represents the most straight forward approach and oxidative coupling between the thiol modified siRNA and the thiol group of cysteine in the protein can be achieved by simply incubating the mixture for 1 h at 40° C. with a thiol cross-linking agent diamide (Sigma), Scheme 1 (FIG. 1). This procedure was used to couple siRNA to both transportan and penetratin (SEQ ID NO: 2).

The more common approach is to activate one of the thiol containing components with a pyridylsulfide before addition of the second thiol component. This allows specific heterodisulfide formation without concomitant homodisulfide formation of the peptide or oligonucleotide component. The reaction is carried out in PBS and is very rapid, Scheme 2 (FIG. 2).

The above coupling chemistry can also be carried out with 3′- or 5′-amino modified siRNA which commercially have been available for a number of years. Here, the primary amine group at the 3′- or 5′ is reacted with an active N-hydroxy succinimide group a hetero-functional coupling reagent SPDP, to produce 2-pyridyl disulfide activated siRNA which then reacts with the thiol group present in the peptide, Scheme 3 (FIG. 3). The use of SPDP is one way of coupling on to the lysine residues of peptides like antennapedia.

A plurality of siRNA's may be coupled on to the antennapedia protein by coupling onto the amine of lysine residues. The simplest approach would be to obtain a suitably modified 3′- or 5′-siRNA, namely one with a terminal carboxylic acid group which could be activated using carbodiimide/N-hydroxy succinimide chemistry to give an ‘activated ester’. Coupling of this ‘activated’ siRNA to the amine groups of lysines on antennapedia (forming a peptide bond) would be achieved in PBS by simply stirring the two components at room temperature for 1 h, Scheme 4 (FIG. 4).

If a suitably functionalised siRNA is difficult to obtain then the antennapedia protein may be thiolated using the heterobifunctional crosslinking agent SPDP (see above) to convert the lysine residues on antennapedia to thiols. Once modified with SPDP, the protein can be treated with DTT (or another disulfide reducing agent) to release the pyridine-2-thione leaving group and form the free sulfuhydryl (thiol). This terminal —SH (thiol) group can then be used to link onto the —SH group on siRNA through a disulfide linkage, Scheme 5 (FIG. 5).

In an alternative approach the 3′- or 5′-amine terminated siRNA may be converted into a carboxylic acid by reacting with glutaric anhydride. The amine group will ring open glutaric anhydride at room temperature, forming an amide linkage and liberating a carboxylic acid, this is standard chemistry. This siRNA is now terminated with a carboxylic acid group which can be converted into an ‘active ester’ (see Scheme 4, FIG. 4) and coupled onto the lysine residues of antennapedia, Scheme 6 (FIG. 6).

Although the invention has been described with reference to the above examples, it will be understood that modifications and variations are encompassed within the spirit and scope of the invention. Accordingly, the invention is limited only by the following claims.

What is claimed is:

1. A method of producing a cell permeable nucleic acid molecule conjugate comprising conjugating a nucleic acid molecule with a cell-penetrating peptide (CPP), the CPP comprising a homeodomain of an antennapedia homeotic transcription factor protein (Antp), or functional fragment thereof.

2. The method of claim 1, wherein the conjugating comprises contacting the nucleic acid molecule, the nucleic acid molecule comprising a thiol group at a 3′ or 5′ end of the nucleic acid molecule, with the CPP, the CPP comprising an amino acid residue including a thiol group, wherein the contacting is performed in the presence of a thiol crosslinking agent.

3. The method of claim 2, wherein the amino acid residue is a cysteine residue.

4. The method of claim 2, wherein the thiol crosslinking agent is diamide.

5. The method of claim 2, wherein the thiol group of the nucleic acid molecule is contacted with a pyridyl sulfide before contacting the nucleic acid molecule with the CPP.

6. The method of claim 1, wherein the conjugating comprises:
a) contacting the nucleic acid molecule, the nucleic acid molecule comprising an amine group at a 3′ or 5′ end of the nucleic acid molecule, with an amino and thiol reactive heterobifunctional crosslinking agent; and
b) contacting the nucleic acid molecule of (a) with the CPP, the CPP comprising an amino acid residue including a thiol group, wherein the contacting is done in the presence of a thiol crosslinking agent.

7. The method of claim 6, wherein the crosslinking agent of (a) is N-succinimidyl-3-(2-pyridyldithio)propionate (SPDP).

8. The method of claim 6, wherein the crosslinking agent of (b) is diamide.

9. The method of claim 6, wherein the amino acid residue is a cysteine residue.

10. The method of claim 1, wherein the conjugating comprises contacting the nucleic acid molecule, the nucleic acid molecule comprising an ester at a 3′ or 5′ end of the nucleic acid molecule, with the CPP, the CPP comprising an amino acid residue including an amine group.

11. The method of claim 10, wherein the amino acid residue is a lysine residue.

12. The method of claim 1, wherein the conjugating comprises:
a) contacting the CPP, the CPP comprising an amino acid residue comprising an amine group, with an amino and thiol reactive heterobifunctional crosslinking agent;
b) contacting the CPP of (a) with a disulfide reducing agent; and
c) contacting the CPP of (b) with a nucleic acid molecule, the nucleic acid molecule comprising a thiol group at a 3′ or 5′ end of the nucleic acid molecule, wherein the contacting is performed in the presence of a thiol crosslinking agent.

13. The method of claim 12, wherein the crosslinking agent of (a) is N-succinimidyl-3-(2-pyridyldithio)propionate (SPSP).

14. The method of claim 12, wherein the disulfide reducing agent of (b) is dithiothreitol (DTT).

15. The method of claim 12, wherein the crosslinking agent of (c) is diamide.

16. The method of claim 12, wherein the amino acid residue is a lysine residue.

17. The method of claim 1, wherein the conjugating comprises:
a) contacting the nucleic acid molecule, the nucleic acid molecule comprising an amine group at a 3′ or 5′ end of the nucleic acid molecule, with a reagent to convert the amine group to a carboxylic acid;
b) contacting the nucleic acid molecule of (a) with a second reagent to convert the carboxylic acid to an ester; and
c) contacting the nucleic acid molecule of (b) with a CPP, the CPP comprising an amino acid residue including an amine group.

18. The method of claim 17, wherein the reagent of (a) comprises glutaric anhydride.

19. The method of claim 17, wherein the amino acid residue is a lysine residue.

20. The method of claim 1, wherein the nucleic acid molecule is RNA or DNA.

21. The method of claim 1, wherein the nucleic acid molecule is RNA.

22. The method of claim 1, wherein the RNA is siRNA.

23. The method of claim 1, wherein the CPP comprises at least 5 contiguous amino acid residues of sequence SEQ ID NO: 1 from residue 283 to 356.

24. A cell permeable nucleic acid molecule conjugate produced by the method of claim 1.

25. A cell permeable nucleic acid molecule conjugate comprising:
a) a nucleic acid molecule; and
b) a cell-penetrating peptide (CPP), the CPP comprising a homeodomain of an antennapedia homeotic transcription factor protein (Antp), or functional fragment thereof.

26. The conjugate of claim 25, wherein the CPP comprises at least 5 contiguous amino acid residues of sequence SEQ ID NO: 1 from residue 283 to 356.

27. The conjugate of claim 25, wherein the nucleic acid molecule is DNA or RNA.

28. The conjugate of claim 25, wherein the nucleic acid molecule is a double stranded RNA molecule.

29. The conjugate of claim 25, wherein the nucleic acid is an siRNA.

30. The conjugate of claim 25, wherein the conjugate comprises a plurality of nucleic acid molecules.

31. A method of introducing a cell permeable nucleic acid molecule conjugate into a cell comprising:
a) producing a cell permeable nucleic acid molecule conjugate using the method of claim 1; and
b) contacting a cell with the conjugate of (a), thereby introducing the cell permeable nucleic acid molecule conjugate into the cell.

32. The method of claim 31, wherein the contacting is performed in vivo or in vitro.

33. The method of claim 31, wherein the nucleic acid molecule conjugate comprises a dsRNA.

34. The method of claim 33, wherein the dsRNA is an siRNA.

35. A method of treating a subject in need thereof by administering to the subject the cell permeable nucleic acid molecule conjugate of claim 25.

36. A pharmaceutical composition comprising the cell permeable nucleic acid molecule conjugate of claim 25.
